(19) United States
(12) Patent Application Publication
(10) Pub. No.: US 2007/0172941 A1
(43) Pub. Date: Jul. 26, 2007

(54) Disposable vessels or tips having ultra-thin areas therein, and methods for manufacture of same

(76) Inventors: Amir Porat, Savyon (IL); Moshe Gombinsky, Bat Yam (IL); Agmon Porat, Savyon (IL)

Correspondence Address: EDWARD LANGER;c/o SHIBOLETH YISRAELI ROBERTS ZISMAN & CO., 1 PENN PLAZA-SUITE 2527, NEW YORK, NY, 10119, US

(21) Appl. No.: 11/338,891

(22) Filed: Jan. 25, 2006

(51) Int. Cl. C12M 3/00

(52) U.S. Cl. 435/287.2; 264/211

(57) ABSTRACT

The invention discloses disposable polymeric vessels or tip products, having a thin area upon at least a portion of the vessel or tip, for use in medical and scientific laboratories. The thin area has a thickness of less than 100 microns. Examples of such products are test-tubes, microtubes and multiwell plates. The thin area is transparent, and is advantageous for performance of optical assays, and in performance of attraction and transfer of magnetic particles. Methods for manufacturing the polymeric vessel and tip products are disclosed.

### FIELD OF THE INVENTION

The present invention generally relates to disposable plastic-ware for scientific laboratories. More specifically, the invention discloses tips, test-tubes, and multiwell plates, having an ultra-thin area of wall with a maximal thickness of 100 microns in that area. Methods for the manufacture of such products are additionally disclosed.

### BACKGROUND OF THE INVENTION

Magnetic particles are used for a variety of separation, purification, and isolation techniques in connection with chemical or biological molecules. In those techniques, a magnetic particle is coupled to a molecule capable of forming a specific binding (“affinity binding”) with a target molecule thought to be present in a biological sample. The magnetic particle is then brought into contact with the biological sample and a magnetic field is applied in order to isolate the biological molecules that are bound to the magnetic particles.

Magnetic microparticles or nanoparticles, are used for instance, to bind DNA molecules, proteins, cells, and sometimes subcellular fragments. In recent years, magnetic microparticles have been used as solid phase for chemical synthesis. Microparticles are in the size of 0.5-10 micron while nanoparticles are 0.05-0.3 micron.

Various devices and methods have been developed in order to separate and transfer magnetic particles after they have bound with their target molecules. Generally, the available methods fall under two categories:

In the first method, the biological fluid and the magnetic particles are placed within a suspension vessel such as a microtube. A magnet is then brought into contact with the outside wall of the microtube, externally to the microtube. The magnetic particles and their bound target molecules, move towards the magnet, and the unwanted supernatant can be removed, while the magnetic particles remain localized on a specific spot of the side or bottom wall of the microtube, due to their attraction to the external magnet.

Alternatively, when many samples need to be treated, the biological fluid is placed within a multiwell plate. A lengthened magnet is then placed externally beneath the multiwell plate, and the magnetic particles move towards the magnet, thus becoming localized at the bottom of the multiwell plate. The remaining liquid is removed out of the vessel via aspiration.

In the second method, a magnetic rod is immersed in the biological sample. This allows better contact between the magnet and the magnetic particles, since there is no side-wall present between the magnet and the micro-particles which would weaken the magnetic field, therefore a higher yield of transferred magnetic particles can be obtained. The magnetic rod with the captured particles is then moved into another vessel. Since the particles are readily transferred to a second vessel, removal of the unwanted supernatant in the source vessel is unnecessary, and one step in the process has been saved. After the transfer is performed, the particles can be moved to one or more additional vessels or returned to the source vessel, as necessary.

Hand-held devices have been designed which terminate in a retractable cylindrical magnet. The magnet is covered with a protective plastic tip, in order to prevent contamination. The covered magnet is immersed in the biological sample, and then the covered magnet is moved into a second microtube containing fresh liquid. The magnet is retracted from within the tip, and the particles that were attached to it become released from the tip into the fresh liquid. The protective tip can be discarded after a single use, or re-used.

U.S. Pat. No. 6,468,810 to Korpela describes one such device, and adds a protective tip made from a flexible material. When the magnet is exposed from within its housing, it presses on the tip causing the material of the tip to stretch so that at the point of the tip, the material becomes somewhat thinner, allowing closer proximity at the thinned area, between the magnet and the magnetic particles. Korpela suggests use of silicone rubber, of a thickness of 0.1-1 mm (which equals 100-1000 microns). A tip having the minimal thickness suggested (0.1 mm, equaling 100 microns) still interferes with transmission of the magnetic field through the tip. This interference results in a visible quantity of magnetic particles remaining behind in the supernatant after the transfer, lowering the yield of particles transferred and thus recovered. This is problematic especially in such cases where the target molecule is difficult to obtain. Additionally, the tip of Korpela stretches to varying degrees at different areas upon the tip, and the degree of stretching obtained depends on the pressure applied, therefore optimal transfer results may not be repeatable.

The need for a thin protective tip on a magnetic device was suggested in U.S. Pat. No. 6,409,925 to Gombinsky et al.

It would be advantageous if test-tubes, microtubes and protective tips would be designed to obtain maximal attraction and transfer high yield of magnetic microparticles.

Microwell plates for use in optical bioassays have light-transparent walls forming the bottom walls of the wells. These light-transparent walls are thin and resemble an optical lens, since they permit transmission of light at desired wavelengths, and absorb light at undesired wavelengths. The light-transparent walls should preferably be as thin as possible in order to allow maximal transmission of light at the desired wavelengths, through the walls.

Microwell plates are also known as “microtiter plates”, “microplates”, “nano-plates” and “deep well plates”.

Manufacture of microwell plates having light-transparent bottom walls is not simple. US 2004/0020595 to Khan et al. describes making an upper frame of opaque polymer forming the side walls of the wells, then applying adhesive to the bottom of the frame to bind a single transparent panel, which acts as the transparent bottom wall of all the wells. The adhesive used is a light-curable adhesive which, according to Khan et al., better seals the wells than prior art adhesives. Khan et al. is limited in that the adhesive must be placed exactly on the side-wall frame, and be prevented from leaking into the wells.

US 2002/0022219 to Clements et al. eliminates the adhesive by including infra-red absorbent particles in the upper well-forming plate material, then after the upper plate is contacted with a lower transparent plate, infra-red radiation is applied to heat and bind the two plates covalently. This method of manufacture is not cost-effective.

These prior art microwell plates suffer from the disadvantage of having a sealed underside so that the sidewalls of the wells cannot be reached from below. Should such a microwell plate be used for magnetic particle separation, a magnet cannot be placed externally upon a side wall, since only the bottom wall is accessible from beneath the microwell plate. It is common practice in magnetic particle separation to place multiple samples in a microwell plate, and to introduce a magnetic plate with multiple lengthened magnets below the microwell plate so that each of the magnets protrudes into the space between four adjacent wells. Thus the magnetic particles from four neighboring samples can be attracted towards their respective side walls with a single magnet. This cannot be done using the microwell plates described above, since the wells are sealed by a single panel which forms a continuous bottom wall, which prevents access to the side walls sealed within.

EP 1348533 to Craig et al. describes a method for fabrication of a multi-well plate by injection-compression, in which a mold cavity is adjusted to allow multiple rounds of injection of molten material, which undergoes compression. Molten polymer thus forms the entire plate, including the transparent bottom wall (clear lens wall). The resultant bottom lens wall tends to be 100-375 micron thick. The method of Craig et al. eliminates the need of attaching a thin glass panel as previously described.

However, EP 1348533 suffers from the disadvantage that the technology available at present does not allow injection of polymer into a mold designed to produce a product having walls thinner than 150 micron. It is impossible to inject polymer into a space smaller than or equal to 100 microns, due to streaming problems that prevent complete filling. EP 1348533 is therefore limited in the thickness of the transparent bottom walls, and cannot be used to obtain walls having a thickness of less than 150 microns.

Microwell plates are available in coated form, in which the inner surface of the wells is coated with antigens or antibodies, which adsorb to the plastic surface of the wells in a hydrophobic interaction with the plastic matrix of the microwell plate. Streptavidin-coated microplates are widely used due to a high affinity interaction between streptavidin and biotin, allowing use of streptavidin-coated microplates to immobilize various biotinilated biomolecules.

In manufacture of most of the commercially coated plates, coating protein is applied at an amount appropriate for covering most of the well surface, necessitating for instance, application of up to 300 microliters of coating protein per well. Coated microplates are therefore more expensive to manufacture than non-coated plates. It would be advantageous to use smaller amounts of coating materials to coat the wells, for a cost-efficient coating process.

It would be desirable to have an efficient method of manufacturing a microwell plate having a thin transparent bottom wall of a thickness of less than 100 microns, which would be ideal for magnetic particle separation. Such a microwell plate should be economical to produce, and should provide the user with access to the side walls for optional introduction of a magnet into the area between adjacent wells. Such a plate could additionally be used for optical biological and chemical assays, having a thin transparent bottom wall, and optical reading of the results would be more accurate.

It would also be desirable to be able to coat the wells of the microwell plate with considerably lower amounts of coating material compared to prior art methods of manufacture of coated plates.

### SUMMARY OF THE INVENTION

The present invention discloses disposable polymeric vessels or tip products, having an ultra-thin area upon at least a portion of the vessel or tip. The thin area has a thickness of less than 100 microns, such as 50-80 microns, more preferably 25-50 microns, and most preferably 5-25 microns. The present invention includes multiwell plates having at least one thin area in a portion of each well. Novel methods of manufacture of these polymeric vessels and tips are disclosed.

In the present invention, the term “microtube” or “micro-tube” refers to a disposable polymeric test-tube able to contain a volume of up to approximately 2.5 cc within. Examples of such microtubes are those manufactured by Eppendorf®, having a volume of either 2 cc, or 2.5 cc, with tapered side-walls which meet to form a point, and an attached flip-type lid. Other microtubes, usually termed “microtainers”, have supporting external side-walls, allowing them to stand independently without the need for a test-tube rack. Microtainers often have removable covers.

The term “test-tube” is intended to include test-tubes of any size. The test-tubes may optionally include removable covers.

Accordingly, it is a principal object of the invention to provide a method of manufacture of a disposable vessel or tip product, having a thin area with a thickness of less than 100 microns, present in at least one portion of said vessel or tip product, said method comprising the steps of:
a. providing a mold shaped to form the image of the walls of said product;

said mold including a pin component being the negative of the interior of said vessel or tip product; said mold further including a first opening for allowing introduction of molten polymer and a second opening for formation of a thin area of said vessel or tip product;
b) placing a thin film of polymeric material having a thickness of less than 100 microns, in contact with said second opening of said mold, for forming the thin area of said vessel or tip product;
c) introducing molten polymer into said mold;
d) allowing sufficient time for cooling of said polymer and for fusing of said polymer and thin film to an integral product;
e) optionally, removing said pin component;
f) optionally, cutting excess film projecting from the outside of said vessel or tip product;
g) removing said mold to obtain a vessel or tip product having a thin area therein.

According to one embodiment, at least one portion of the thin film of polymeric material is coated with a biological or chemical material prior to contacting the film with the mold. Examples of biological materials may be antibodies or antigens.

The present invention also provides a method of manufacture of a disposable vessel or tip product, having a thin area with a thickness of less than 100 microns, present in at least one portion of said vessel or tip product; comprising the steps of:
a) providing a mold shaped to form the image of the walls of said product;
said mold including a pin being the negative of the interior of said vessel or tip product;
b) introducing molten polymer into said mold;
c) compressing a portion of said molten polymer using said pin, to obtain a thin area on said vessel or tip product at the site of said compression;
d) cooling said polymer;
e) removing said mold to obtain said vessel or tip product.

In preferred embodiments of the invention, the vessel or tip is selected from: a test-tube, a multiwell plate, and a tip for a hand-held or automated magnet device for transfer of magnetic particles.

Preferably, the products of the invention are formed from an organic polymer such as: polypropylene, polycarbonate, nylon, Teflon™, and polystyrene. Further features and advantages of the present invention will become more readily apparent and understood from the detailed description of the invention provided below.

### BRIEF DESCRIPTION OF THE DRAWINGS

For a better understanding of the invention with regard to the embodiments thereof, reference is made to the accompanying drawings, in which like numerals designate corresponding elements or sections throughout and in which:

FIG. 1a-1c illustrates steps 1-3 in a manufacturing process according to one embodiment of the invention;

FIG. 2a-2c illustrates steps 4-6 of the same process;

FIG. 3a-3b illustrates two steps of a second manufacturing process according to an alternative embodiment of the invention;

FIG. 4 illustrates the definition of Young's modulus and bulk modulus;

FIG. 5 is a perspective view of a multiwell plate according to the invention;

FIG. 6 is a perspective view from below of the multiwell plate, in which the, the accessibility of the wells from beneath the plate is apparent;

FIG. 7 illustrates a strip of wells manufactured according to the invention, with FIG. 7a showing a view from above, and FIG. 7b showing a view from below including well bottoms; and

FIG. 8 illustrates various possible orientations of the thin-walled areas upon test tubes or upon the wells of a multiwell plate.

### DETAILED DESCRIPTION OF PREFERRED EMBODIMENTS

The invention discloses disposable polymeric vessels or tip products, having an ultra-thin area upon at least a portion of the vessel or tip. The thin area has a thickness of less than 100 microns, such as 50-80 microns, more preferably 25-50 microns, and most preferably 5-25 microns. The technology for production of such thin areas in disposable polymeric test tubes and tips has not been available till now.

In prior art, the thinnest tips available for hand-held retractable magnetic devices or for micropipettors, are approximately 150-300 microns thick.

In contrast, the present invention discloses vessels and tips having a thin area, which can have a thickness of less than half the thickness of a sheet of paper (paper has a thickness of about 70 microns).

The thin area upon the vessel or tip is ideal for attraction and transfer of magnetic particles, since the thin area allows closer proximity of the magnet and a stronger magnetic field to reach the particles than in the thicker prior art test-tubes and thicker prior art tips for hand-held magnetic devices. The yield of magnetic particles attracted and transferred using the invention is therefore higher than in prior art.

Additionally, the invention allows manufacture of multiwell plates having at least one thin area in a portion of each well. The thin area is highly transparent, and therefore allows optical assays to be accurately performed. The thin area additionally allows attraction of magnetic particles through the thin area and grants a high yield of particles attracted. The thin area in each well is easily accessible from beneath the multiwell plate, allowing introduction of a magnet into the space between adjacent wells and further allowing placement of a magnetic plate beneath the well bottoms.

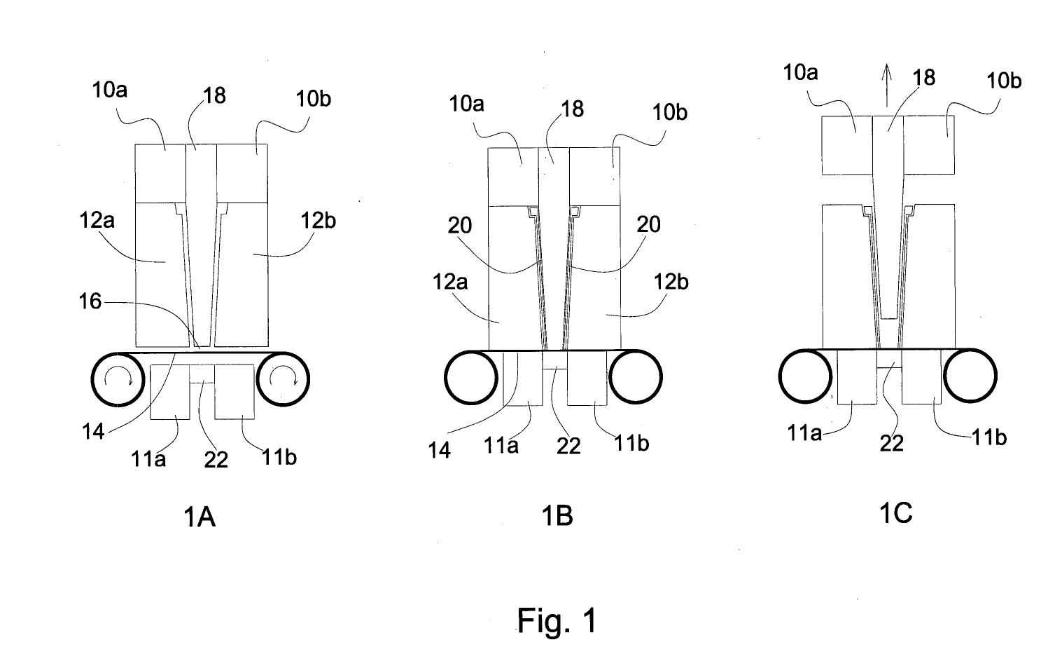

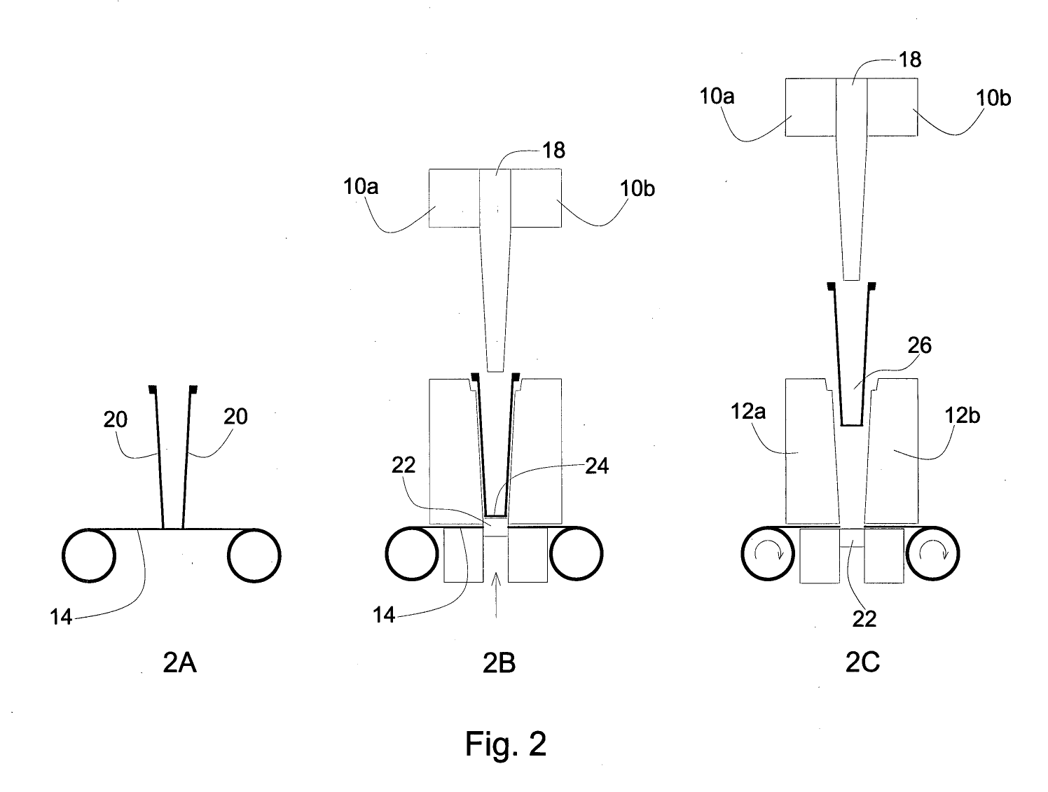

In FIGS. 1 and 2, a method of manufacture of a test-tube, microtube, a tip, or a single well within a multiwell plate is illustrated and described.

Referring to FIG. 1a, mold components 10a, 10b and 12a, 12b are present within a molding machine. The mold components are shaped to form the side-walls of a microtube product, or to form the side-walls of an individual well within a multiwell plate. Alternatively, the mold forms the side-walls of a tip product, or a test-tube. Mold components 10a, 10b, 11a, 11b, 12a, 12b, and 22 form the negative image of these side-walls. Pin component 18 is the negative image of the interior of the product, therefore acts to form the cavity present in the interior of the product. A thin film 14 of polymeric material contacts an opening 16 in the mold. The film 14 has the final thickness desired for the thin area of the product, namely less than 100 microns.

Referring to FIG. 1b, the mold components (10a,b; 12a,b; 11a,b, 22 and 18) are brought together, and molten polymer is injected into the mold components, to form the side walls 20 of the product. The molten polymer reaches and contacts the thin film 14, and melts the thin film 14 at the points of contact. Thus as the polymer cools, the side-walls 20 and the thin film 14 which will act as the bottom wall, become integrated to form a single part. Thus the side walls and bottom walls fuse to a single unit, which appears seamless. The unit is capable at this stage of, for instance, containing within a liquid, without leakage occurring.

Referring to FIG. 1c, after the polymer injected has cooled, the pin 18 and mold components 10a,b are removed from the mold.

Referring to FIG. 2a, the vessel or tip product is shown with the mold stripped away, for illustrative purposes. The product 20 formed is attached to the film 14.

Referring to FIG. 2b, mold component 22 moves upwards and acts as a cutting-block in addition to its function as a mold component. The mold component 22 has sharpened cutting edges at its extremities, and when moved upwards to enter the mold cavity, acts to sever the thin-filmed bottom wall 24 from the remainder of the sheet of thin film 14, leaving behind the excess film that projected from outside the product.

The upward movement of mold component 22 also acts to eject the product from within the mold cavity.

Referring to FIG. 2C, product 26 is now released, after mold components 10a,b, and 18 have risen out of the center of the mold, and mold component 22 has forced the product 26 out of the mold.

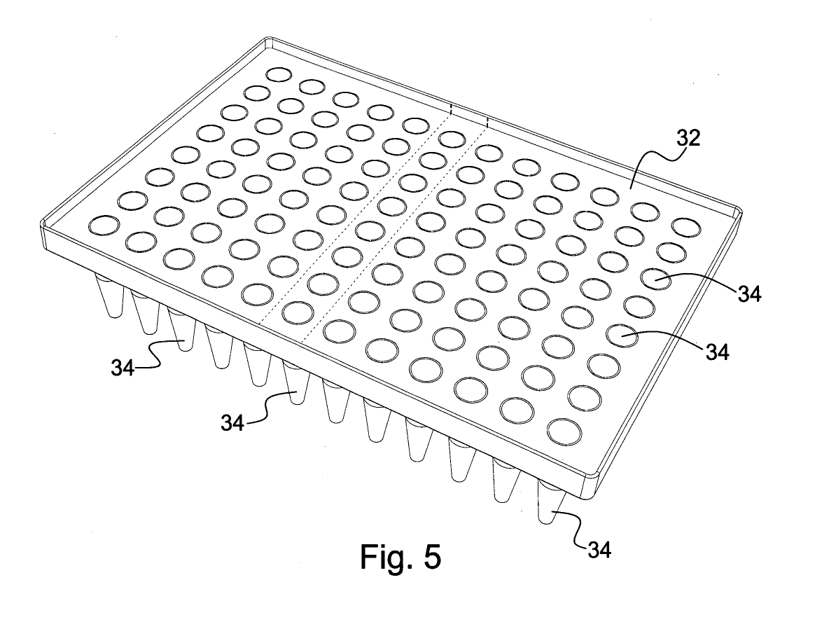

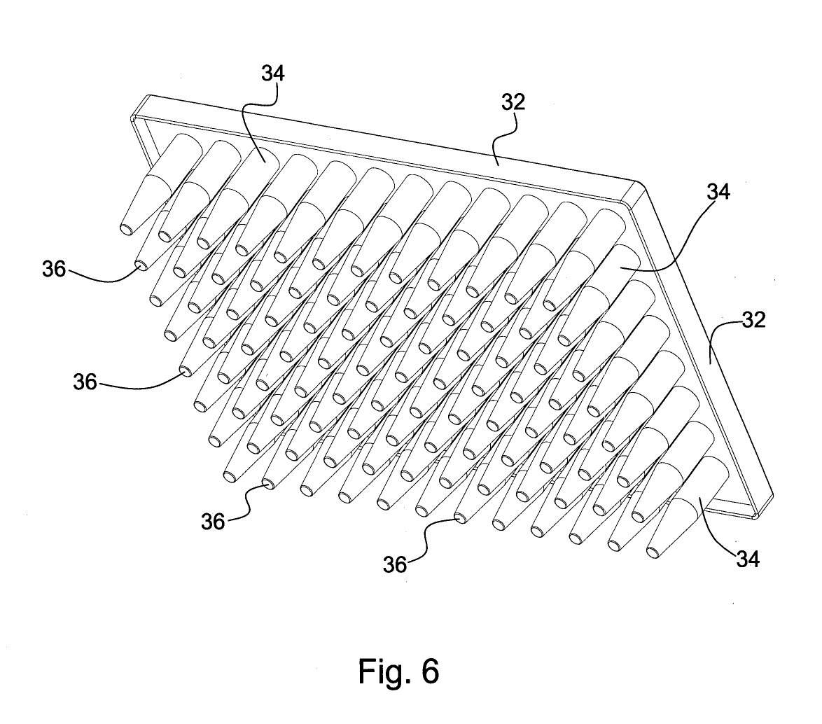

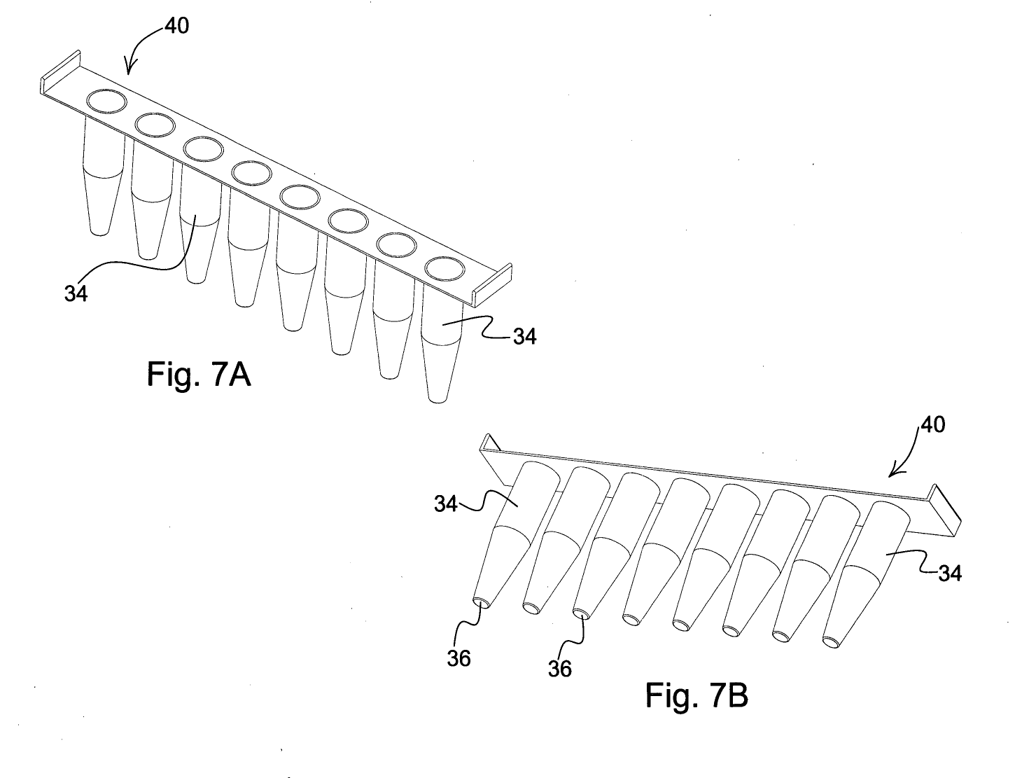

When the final product is a tip, a microtube or a test-tube, product 26 represents the final product, having a thin bottom wall or a thin side wall. When the final product is a multiwell plate, the mold may be shaped to form the number of wells present in the final microwell plate, shown in FIGS. 5 and 6 (described hereinbelow). Alternatively, the mold may form a strip of wells as shown in FIG. 7. (described hereinbelow).

In an automated process, a multitude of cavity molds having film contacting their openings, such as described in relation to FIGS. 1 and 2, can be arranged within the mutimold cavity of a molding machine, and molten polymer can be injected simultaneously in an automated process that is timed to allow cooling of polymer, and cutting of the products.

To initiate each subsequent cycle of manufacture, mold components are rejoined together, and film 14 is rolled to advance such than an unused portion of film contacts the mold components (10a,b, 11a,b, 12a,b, 18 and 22), allowing each subsequent injection of molten polymer.

Preferably, the molten polymer is an organic polymer is selected from: polypropylene, polycarbonate, nylon, Teflon™, and polystyrene.

In one embodiment, the thin film is polypropylene, at a thickness of approximately 25 microns.

In certain embodiments, a multiwell plate is formed having a large number of wells per plate. In such case, the lower peripheries of the bottom walls of adjacent wells may be joined together, with little or no space present beneath the plate between adjacent wells. Excess polymer may therefore not always be present on the external side of the thin bottom walls of the wells, since most of the thin film has been used to form a multitude of wells. Mold component 22 may therefore not be needed to sever excess polymer between all adjacent wells, and may, for instance, cut groups of wells instead of individual wells.

Though in FIGS. 1 and 2 the thin-walled area is illustrated beneath the mold, so that it will form the bottom wall of the vessel or tip product, this placement is for illustrative purposes only. The thin area may be placed at any point upon the product, with the mold being open at the appropriate place, and thin polymer film contacting the opening. The thin area may be placed upon a side-wall, a vessel cover, or a combination of these locations. Moreover, more than one thin area may be present in a single vessel or tip.

The manufacturing process previously described allows the advantage of easily treating the bottom walls of a multiwell plate. It is common practice to coat multiwell plates after their manufacture, with biological or chemical materials, such as biological blocking material, antibodies, antigens, florescent material, and reagents that can undergo a color change. In prior art, care must be taken to ensure the coating reaches all surfaces of interest within the vessel or well, therefore relatively large amounts of coating material are typically used. In contrast, the present invention allows the bottom walls to be effortlessly coated by applying the coating onto the thin film before the film is placed in contact with the mold. When the coating is in liquid form, the entire sheet of film can be easily immersed in the liquid coating. The coated film is then dried and placed adjacent to the mold. Alternatively, the coating can be applied using a highly controlled injection process which creates coated zones of predetermined size, upon areas of the film, which ultimately become the thin areas present in the final product. This can minimize the amount of coating material utilized.

In one preferred embodiment, the coating used is streptavidin.

According to certain embodiments, the thin film can be colored with a coloring agent or dye, prior to its placement in contact with the mold components. The resultant bottom walls, which originate in the thin film of polymer, have a first color, while the side walls, formed from molten polymer, are either colorless or have a second color.

Though the thin film has been described in relation to FIGS. 1 and 2 to be present as a roll of film, which necessitates excess film being cut off at the end of the process, optionally the thin film can be precut into pieces sized to fit the opening of the mold. In such case, in a production line, each piece of film is applied separately to an appropriate mold opening.

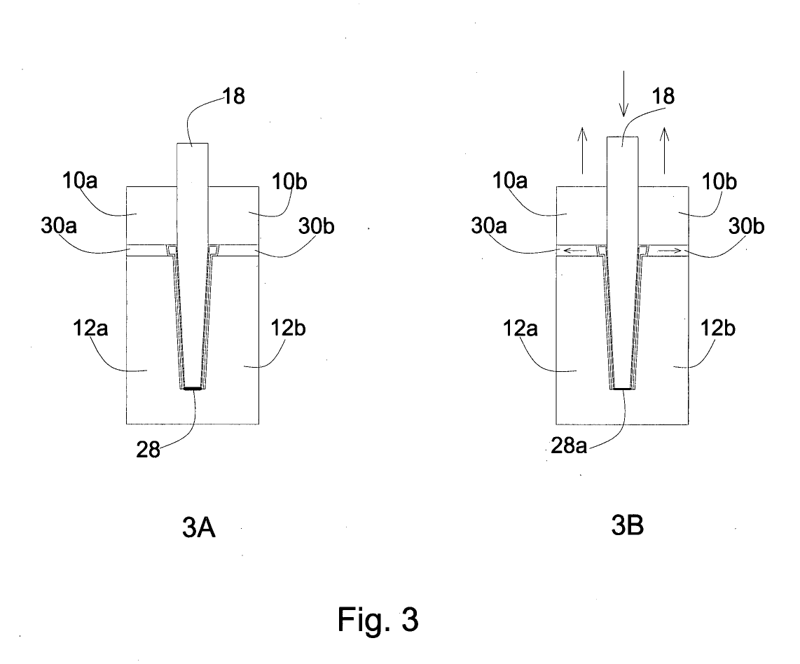

In FIG. 3, an alternative method of manufacture is described for a vessel or tip product having a thin area. Referring to FIG. 3A, mold contains additional mold components 30a, 30b. After pin 18 is placed within assembled mold components (10a,b, 12a, 12b, and 30a,b) molten polymer is injected into the mold until to a temporary wall thickness of 200-300 micron is reached at bottom wall 28. Referring to FIG. 3B, a predetermined force is then applied to pin 18 in the downward direction shown by arrow, before molten polymer cools completely, to compress molten polymer present at the base 28a of the pin into a thin area. Small amounts of excess molten polymer seep out towards mold components 30a,b and 10a,b, as shown by horizontal arrows, and similarly seep upwards around the pin 18 as shown by the vertical arrows. The bottom wall formed will therefore have a thin area having a maximal thickness of 100 microns. The final thickness of the thin area is determined based on the force applied by pin 18 on the polymer, and additionally depends on the characteristics of the polymer used.

Should a thin area be desired in a side wall, or on a vessel cover, the mold will be designed in the appropriate orientation, so that pin 18 descends towards the area desired as the thin area.

This method may be automated, and may occur within a molding machine or after injection using a specific tool.

Preferably, the molten polymer is an organic polymer selected from: polypropylene, Teflon™, and polystyrene.

In order to determine the amount of force necessary for the pin to descend in order to compress the molten polymer to achieve the desired thickness, Young's Modulus of material, or the bulk modulus of material are considered. These describe the relationship between the compression of a material, and the stress placed upon the material.

The bulk elastic properties of a material determine how much it will compress under a given amount of external pressure. The ratio of the change in pressure to the fractional volume compression is termed the bulk modulus (B) of the material:

$$\frac{Δ P}{Δ V / V} = B$$

$$Δ P = applied pressure$$

$$Δ v = change in volume$$

$$V = volume$$

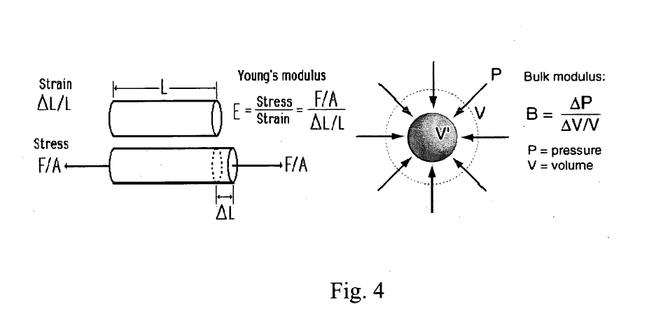

Referring to FIG. 4, Young's modulus is illustrated. Young's modulus is similar to the bulk modulus, however it pertains to a single dimension.

For example, polypropylene has a bulk modulus of approximately 1.5525×109 Pa. Therefore, in order to achieve a change in thickness from 300 micron to 30 micron, it is necessary to apply the following force:

$$Δ p = \frac{270}{30} * 1.5525 \times {10}^{9} = 13.97 \times {10}^{9} Pa$$

An Example of the Elastic Properties of Materials

| | | Young's | Ultimate Strength | Yield Strength |
|---|---|---|---|---|
| | Density | Modulus | Sᵤ | S_{y} |
| Material | (kg/m³) | 10⁹ N/m² | 10⁶ N/m² | 10⁶ N/m² |
| Polystyrene | 1050 | 3 | 48 | . . . |

| Mechanical Properties | Norm | Unit | PP | PP-R | HiPro |
|---|---|---|---|---|---|
| Tensile strength at yield (σₛ)/ | ISO 2039-1 | MPa | 75 | 45 | 110 |
| Rockwell | | | | | |
| Flexural strength (σ_{B3.5%}) | ISO 178 | MPa | 35 | 20 | |
| Modulus of elasticity (E_{I}) | ISO 527 | MPa | 1350 | 700 | 6500 |

Referring to FIG. 5, there is shown a multiwell plate, whose wells were formed according to the invention. Support frame 32 surrounds wells 34 and joins the wells at their upper peripheries. Support frame 32 is polymeric, and is usually cast separately from wells 34. Common sizes of multiwell plates are 96-well (8×12 wells) and 384-well (16×24 wells), though other size plates are possible.

Due to the above-described methods of manufacture, a multiwell plate is obtained in which each well has at least one thin area. The thin areas may be accessed from beneath the multiwell plate, since support frame 32 is open and bottomless, as best seen in FIG. 6.

In contrast, prior art multiwell plates having transparent bottom walls are closed from beneath, since the bottom walls are almost always formed from a single plate.

Referring to FIG. 6, the multiwell plate is shown from below. Wells 34 have transparent thin-walled bottom walls 36 of less than 100 microns thickness, allowing optical assays to be performed in the multiwell plate.

When a coated multiwell plate is produced according to the invention, the coating material present upon the multiwell plate, may undergo an interaction with the liquid contents of the wells. This interaction may be optically detectable using an ELISA plate reader, or a similar instrument. The presence of thin bottom walls that do not deflect light ensures maximal transmission of illumination through the plates, and thus highly accurate optical readings. Similarly, fluorescence and phosphorescence can be readily and accurately detected due to the thin bottom walls.

Alternatively, the multiwell plate of the invention may be used for magnetic separation of biological materials using magnetic particles. One or more magnets may be introduced beneath the open support frame 32 to access and contact the external side of the thin bottom walls 36 and attract magnetic particles towards the bottom walls 36.

When the thin area is present in the side-wall of each well, it may be advantageous to insert individual magnets into the space between four adjacent wells and attract magnetic particles from four adjacent wells in a single step. A higher yield of particles is obtained than in prior art thicker-walled plates.

Referring to FIG. 7, a strip 40 of wells may be manufactured as a single unit, for assembly into a multiwell dish, or for use in its present condition. Referring to FIG. 7B, each well 34 of the strip 40 has a thin area on its bottom wall 36.

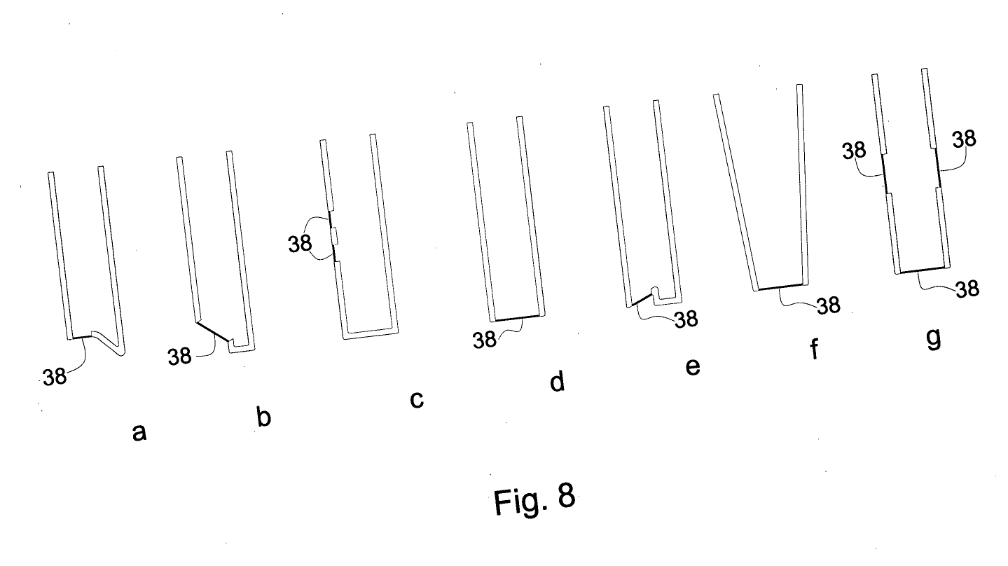

Referring to FIG. 8, various possible orientations of the thin-walled areas 38 upon test tubes or upon the wells of a multiwell plate, are shown in bold. The test-tube may have a bottom wall of specialized shape, formed of an angled portion and a horizontal portion (see a, b, e). The thin area 38 may be present at the horizontal portion (a). The arrangement shown in (a) is advantageous for use in magnetic separation technique, since it allows attraction of magnetic particles at horizontal thin area 38, with undesired supernatant draining downwards to the tapered lower point of the test-tube, for easy removal. Alternatively, the thin area 38 may be present at the angled portion (b, e). Referring to (c), two thin areas 38 are shown upon a side-wall. Referring to (d) and (f), the entire bottom wall is a thin area 38. In (f), side-walls are tapered. Referring to (g), thin areas 38 are present upon opposite side walls, aligned at similar heights upon the side wall. This thin areas in (g) can be easily produced using the method described in relation to FIGS. 1 and 2, with precut pieces of the thin film placed upon the appropriate mold openings instead of a roll of film.

Having described the invention with regard to certain specific embodiments thereof, it is to be understood that the description is not meant as a limitation, as further modifications will now become apparent to those skilled in the art, and it is intended to cover such modifications as are within the scope of the appended claims.

1. A method of manufacturing a disposable vessel or tip product, having a thin area with a thickness of less than 100 microns, said area present in at least one portion of said vessel or tip product, said method comprising the steps of:
a) providing a mold shaped to form the image of the walls of said product; said mold including a pin component being the negative of the interior of said vessel or tip product; said mold further including a first opening for allowing introduction of molten polymer and a second opening for formation of a thin area of said vessel or tip product;
b) placing a thin film of polymeric material having a thickness of less than 100 microns, in contact with said second opening of said mold, for forming the thin area of said vessel or tip product;
c) introducing molten polymer into said mold;
d) allowing sufficient time for cooling of said polymer and for fusing of said polymer and thin film to an integral product;
e) optionally, removing said pin component;
f) optionally, cutting excess film projecting from the outside of said vessel or tip product;
g) removing said mold to obtain a vessel or tip product having a thin area therein.

2. The method of claim 1, wherein said vessel or tip product is a disposable product selected from: a test-tube, a multiwell plate, and a tip for a hand-held magnet device for transfer of magnetic particles.

3. The method of claim 1, wherein said molten polymer is an organic polymer selected from: polypropylene, Teflon™, nylon, polycarbonate, and polystyrene.

4. The method of claim 1, wherein said film of polymeric material is formed of polypropylene, at a maximal thickness of 25 microns.

5. The method of claim 1, wherein said mold is present in a multimold cavity within a machine, and said method is performed in an automated fashion.

6. The method of claim 1, wherein said molten polymer is introduced into said mold via injection.

7. The method of claim 1, wherein at least a portion of said thin film of polymeric material is coated with a biological or chemical material prior to contacting said film with said mold.

8. The method of claim 7, wherein said coated film of polymeric material is cooled during said step of contacting said film with said mold.

9. The method of claim 7, wherein said biological or chemical coating material is selected from: a biological blocking material, an antibody, an antigen, a florescent material, and a reagent that can undergo a color change.

10. The method of claim 7, wherein said coating material is streptavidin.

11. The method of claim 1, wherein said thin film of polymeric material is colored or dyed prior to contacting said film with said mold.

12. A method of manufacturing a disposable vessel or tip product, having a thin area with a thickness of less than 100 microns, said area present in at least one portion of said vessel or tip product, said method comprising the steps of:
a) providing a mold shaped to form the image of the walls of said product;
said mold including a pin being the negative of the interior of said vessel or tip product;
b) introducing molten polymer into said mold;
c) compressing a portion of said molten polymer using said pin, to obtain a thin area on said vessel or tip product at the site of said compression;
d) cooling said polymer;
e) removing said mold to obtain said vessel or tip product.

13. The method of claim 12, wherein said molten polymer is an organic polymer selected from: polypropylene, polycarbonate, nylon, Teflon™, and polystyrene.

14. The method of claim 12, wherein said vessel or tip product is a disposable product for use in a laboratory, selected from: a test-tube, a multiwell plate, and a tip for a hand-held magnet device for transfer of magnetic particles.

15. A disposable polymeric vessel or tip product for use in a laboratory, having a thin area with a thickness of less than 100 microns, said area present on at least one portion of said vessel or tip product.

16. The product according to claim 15, wherein said product is formed of an organic polymer selected from: polypropylene, polycarbonate, nylon, Teflon™, and polystyrene.

17. The product of claim 15, wherein said thin area has a thickness selected from one of the following ranges: 50-80 microns, 25-50 microns, and 5-25 microns.

18. The product of claim 15, wherein said vessel is a test-tube.

19. The product according to claim 18, wherein said test-tube is a microtube, for containing a volume of up to 2.5 cc.

20. The product according to claim 18, wherein said test-tube can contain within a volume of approximately 5 to 500 ml.

21. The product of claim 18, wherein said test-tube has at least two of said thin areas present on said test-tube.

22. The product of claim 18, wherein the position of said thin area is selected from: a side wall of said test-tube, a bottom wall of said test-tube, and the cover of said test-tube.

23. The product of claim 18, wherein said test-tube has a bottom wall comprising an angled portion and a horizontal portion, and one of said angled portion and horizontal portion is a thin area.

24. The product of claim 15, wherein said product is a tip shaped to fit over a retractable cylindrical magnet, said magnet being part of a hand-held magnet device for transfer of magnetic particles.

25. The product of claim 24, wherein said thin area is present at the point of said tip.

26. The product of claim 15, wherein said product is a tip product shaped to fit onto a micropipettor.

27. The product of claim 15, wherein said product is a disposable polymeric multiwell plate, having at least one light-transparent polymeric thin area, present on at least a portion of each of the wells of said plate, wherein said thin area has a thickness of less than 100 microns.

28. The product of claim 27, wherein access is provided from below the plate, to the external surface of the side-walls of the wells of said plate.

29. The product of claim 27, wherein said thin areas are coated with a biological or chemical material.

30. The product of claim 27, wherein said thin areas are colored.

31. The multiwell plate of clam 28, wherein in at least a portion of the wells, the lower peripheries of the bottom walls of adjacent wells are joined together.
